(19) United States
(12) Patent Application Publication
(10) Pub. No.: US 2026/0330424 A1
(43) Pub. Date: Oct. 22, 2026

(54) AUTONOMOUS HEALTHCARE APPOINTMENT MANAGEMENT SYSTEM

(71) Applicant: Oracle International Corporation, Redwood Shores, CA (US)

(72) Inventors: Nathan Smalley, Los Angeles, CA (US); Harshit K. Kalley, Belmont, CA (US); Maxim Romanenko, Altamonte Springs, FL (US)

(73) Assignee: Oracle International Corporation, Redwood Shores, CA (US)

(21) Appl. No.: 19/376,504

(22) Filed: Oct. 31, 2025

Related U.S. Application Data
(60) Provisional application No. 63/791,030, filed on Apr. 18, 2025.

(51) Int. Cl. G06Q 10/1093; G06F 40/205; G16H 40/67

(52) U.S. Cl. CPC G06Q 10/1093; G06F 40/205; G16H 40/67

(57) ABSTRACT

The present disclosure relates to automated routing and scheduling techniques for managing user requests via one or more large language models (LLMs). The disclosed techniques may involve receiving a request from a user in a natural language and parsing user-specific data from a current state. The disclosed techniques may also include predicting a context of the request using pre-historical logs, identifying resources corresponding to the request from a data source, verifying availability of resources for a time frame, determining a schedule for the request using a machine-learning engine, retrieving application programming interfaces (APIs) corresponding to the resources from a meta database, automating request scheduling using the APIs, and calling an execution tool to modify the current state at a user interface. Finally, disclosed techniques may also involve generating a new state at the user interface including an artificial intelligence (AI) generated chat response, a list of flags, and mediated errors.

### CROSS REFERENCE TO RELATED APPLICATIONS

This application claims the benefit of U.S. Provisional Patent Application No. 63/791,030 filed on Apr. 18, 2025. The entire disclosure of the aforementioned application is incorporated by reference herein in its entirety for all purposes.

### BACKGROUND

Healthcare providers often require a treatment plan to deliver comprehensive and personalized care for their patients/subjects. A treatment plan, which may include a detailed profile of a subject's medical history that serves as a roadmap for managing specific medical conditions, illnesses, or injuries. By documenting a subject's medical history, healthcare providers can gain valuable insights into past illnesses, chronic conditions, and previous treatments of a subject. This information can be utilized to make informed decisions about the most appropriate treatment options, preventing adverse reactions, and ensuring the effectiveness of prescribed therapies. A well-structured treatment plan may also facilitate accurate information exchange between healthcare providers and subjects, ensuring efficient allocation of relevant resources by scheduling appointments in a timely fashion.

Most healthcare providers now-a-days rely on computer systems for appointment scheduling, as it not only improves staff productivity by reducing idle time but also ensures that resources are allocated optimally. Additionally, automated reminders may help reduce no-shows, ensuring that appointments and resources are not wasted. Traditionally, such computer systems may rely on manual processes, which are time-consuming and prone to human errors. They also lack real-time data and analytics, making it difficult to quickly adapt to unexpected changes by scheduling optimally. As the number of subjects seeking medical advice grows, traditional systems may not be able to scale efficiently, leading to decreased productivity and resource allocation issues. Moreover, in traditional encounters, providers may not be able to provide a personalized experience, lacking the ability to offer personalized reminders and interactions that may enhance the subject's satisfaction.

The limited and inadept decision-making skills of these computer systems can harm the quality and effectiveness of the treatment plan. Advanced software, particularly those empowered with artificial intelligence (AI), can analyze big electronic medical data quickly and accurately, identifying patterns and insights that might be otherwise missed by providers. This capability allows for more precise and personalized medical treatment plans, according to the need and health state of each subject. AI-powered software can also provide real-time updates and recommendations based on the latest medical research and a subject's health data, ensuring that the treatment plan of a subject remains relevant and effective. Additionally, such software can help healthcare providers anticipate potential complications and adjust the treatment plan proactively, thereby improving subject outcomes.

### SUMMARY

Certain aspects and features of the present disclosure relate to automated routing and scheduling techniques for managing user requests (i.e., appointment requests, asking user specific questions if there is a change in health condition, or dynamically changing schedule of appointments based on user preferences) via one or more large language models (LLMs). The disclosed techniques may involve receiving, via a user interface, a request from a user including user-specific data in a natural language and parsing user-specific data from a current state. The disclosed techniques may also include predicting a context of the request using pre-historical logs, identifying resources from a data source corresponding to the request, verifying availability of resources for a time frame, determining a schedule for the request using a machine-learning engine, retrieving application programming interfaces (APIs) corresponding to the resources from a meta database, automating request scheduling using the APIs, and calling an execution tool to modify the current state at a user interface. Finally, disclosed techniques may also involve generating a new state at the user interface including an artificial intelligence (AI) generated chat response, a list of flags, and mediated errors. The disclosed method may be modeled as an AI agent along with an agent provider service that may provide services to the AI agent and maintain the context between graph executions. The services of the agent provider service may include providing risk management, user interactions, data analysis, etc. The AI agent may use a static directed cyclic graph that includes one or more LLM nodes that are executed in parallel to manage request processing. The AI agent may plan for scheduling of the request and dynamically change the schedule of the request based on different parameters such as subject availability, resource availability, change in wait period, medical emergencies, etc. The current state may indicate a chatbot-interaction between the user and the AI agent at a given instant of time.

The disclosed techniques may generate a treatment plan according to a user's/subject's health condition to schedule the request efficiently. Generating the treatment plan may include parsing user-specific data from a current state, utilizing pre-historical logs and the one or more LLM nodes to collect information about the user, identifying resources from a data source to fulfill the request, verifying the availability of resources for a time frame, and determining a suitable appointment schedule for the request using the machine-learning engine. The LLM nodes may include one or more intelligence nodes that can invoke machine-learning engine one or more times, and one or more execution nodes for using one or more execution tools.

The treatment plan may refer to a plan for appointment scheduling. The treatment plan may include structured data indicating appointments with one or more providers and managing and tracking these appointments in real time. The treatment plan may also incorporate features such as online booking portals, automated reminders to subjects via SMS or email, and tracking in real-time the availability of subjects to reduce no-show rates at healthcare providers. Additionally, the treatment plan may include prioritization protocols to ensure that urgent cases are addressed promptly in real time, yet routine appointments are still efficiently managed to optimize the provider's schedule.

In one aspect, a follow-up request may be made at the user interface to confirm scheduling actions in case of a complex request. The data source may contain one or more resources of health providers/organizations and may enable data exchange across one or more organizations over a cloud network. In another embodiment, the technique may involve using fuzzy logic controls or additional LLMs to reduce waiting time while scheduling user requests.

In yet another aspect, the techniques mentioned in the disclosure may involve an autonomous healthcare appointment management system (AHAMS) that can further be divided into the following aspects: advanced reasoning and function-calling capabilities by leveraging LLMs, an autonomous routing server, an autonomous scheduling server, a chat message synthesizer, an agent provider service, and a healthcare system integration layer. The AHAMS may function as an AI agent that may use a directed cyclic graph. An agent provider service may provide a session-based interface and conversational interface to the AI agent, facilitating conversation with users in natural language. The agent provider service may maintain the current state which may include the session information, including chat message history, feedback metadata, and appointment change lists. The agent provider service may allow users to request multiple appointment changes in a single message and each change may contain a list of fields that are populated once the autonomous scheduling server has completed its tasks.

The LLMs may be used for decision-making, generating chat messages, and selectively invoking customized software routines. The LLMs may be invoked multiple times to complete specific tasks for other components of the AHAMS. The LLMs can detect when software functions may be called and then respond to inputs, allowing an AI agent to select tools that may be used to complete tasks. In API calls, functions can be described, and the LLM can intelligently output a JSON object containing arguments to call these functions. When the LLM responds with an intent to use execution tools, the execution tools may be executed within execution nodes. Execution tools may include checking availability of slots for appointments, scheduling appointments, and then modifying or canceling scheduled appointments dynamically.

In other aspects, the autonomous routing server may be used for classifying content of a user request and routing the user request to relevant LLM nodes. The autonomous routing server may also be used to plan an appointment that may comprise generating a treatment plan for the user based on the availability of resources and other parameters. The autonomous routing server may include two intelligence nodes and one execution node in a graph. The autonomous scheduling server may be triggered by the autonomous routing server once a treatment plan is created. The autonomous scheduling server may consist of four intelligence nodes and an execution node. The autonomous scheduling server may be used to create, cancel, or modify appointments using the execution tools. The intelligence and execution nodes of the autonomous routing and scheduling server may function in parallel to each other for faster computation. The chat message synthesizer is a single intelligence node in the AI agent graph that may understand changes in the state once the graph instance is executed and then subsequently may generate a concise chat reply for a user. If a follow-up information is present, the chat message synthesizer may prompt the user for relevant information. If no follow-up information is present, the chat message synthesizer may produce a user-friendly summary of all actions done during the time a graph's instance was executed.

In some embodiments, a system is provided that includes one or more data processors and a non-transitory computer-readable storage medium containing instructions which, when executed on one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 illustrates an exemplary block diagram of an autonomous healthcare appointment management system (AHAMS) along with an agent provider service in accordance with some aspects of the present disclosure.

FIG. 2 illustrates a block diagram of a healthcare systems integration layer at a cloud network in accordance with some aspects of the present disclosure.

FIG. 3A illustrates a sequence diagram for request routing and scheduling via AHAMS.

FIG. 3B illustrates a block diagram for request routing and scheduling using one or more LLM nodes in accordance with some aspects of the present disclosure.

FIG. 4 shows an exemplary AI agent graph for transitioning between one or more LLM nodes in accordance with some aspects of the present disclosure.

FIG. 5 shows an exemplary user interface illustrating a state at a user device according to some aspects of the present disclosure.

FIG. 6 shows an exemplary treatment plan developed by the AHAMS for a user via a machine-learning engine.

FIG. 7 illustrates an autonomous healthcare appointment management dashboard at a user device with a list of appointments and other details.

FIG. 8 illustrates a flow chart of a method for automating scheduling of healthcare appointments and managing user requests according to some aspects of the present disclosure.

FIG. 9 illustrates an exemplary flowchart for planning and creating an appointment at the AHAMS as an embodiment of the present disclosure.

FIG. 10 depicts a simplified diagram of a distributed system for implementing an embodiment of the present disclosure.

FIG. 11 is a simplified block diagram of a cloud system environment in accordance with certain aspects of various embodiments of the invention.

FIG. 12 illustrates an exemplary computer system that may be used to implement certain aspects of the present disclosure.

### DETAILED DESCRIPTION

The present disclosure relates to an autonomous healthcare appointment management system (AHAMS) for automated routing and scheduling of user requests via one or more large language models (LLMs). The AHAMS can further be divided into the following aspects: advanced reasoning and function-calling capabilities by leveraging LLMs, an autonomous routing server, an autonomous scheduling server, a chat message synthesizer, an agent provider service, and a healthcare system integration layer. The AHAMS may function as an AI agent that may use a directed cyclic graph. An agent provider service may provide a session-based interface and conversational interface to the AI agent, facilitating conversation with users in natural language. The agent provider service may maintain a current state that may contain session information, including chat message history, feedback metadata, and a change in appointments if applicable. The agent provider service may allow users to request multiple appointment changes in a single message and each change may contain a list of fields that are populated once the autonomous scheduling server has completed its tasks.

The disclosed techniques may be used by an AI agent to coordinate and schedule appointments of subjects with providers, via natural language messages as input. In this disclosure, the terms “user,” “subject,” and “patient” are used interchangeably. The AHAMS may autonomously understand the message and can plan and make appointment changes across all healthcare providers, asking users for supplemental information by using various execution tools. The AHAMS may provide several benefits over non-intelligent systems, including understanding messages or requests in a natural language, planning actions, decision-making, and addressing errors or problems by executing appointment-related tasks. The AHAMS may interact with users in a chatbot-style process, modeled as the AI agent and agent-provider service to maintain a context between graph executions. The current state is updated by new messages including conversation and tool outputs. The current state may indicate a chatbot interaction between the user and the AI agent at a given instant of time. The state may further store the chat history and get feedback from the user. The AI agent may use a static directed cyclic graph, with two types of LLM nodes: intelligence node or execution node. The AI agent graph may be executed, and one execution of the agent graph may indicate one agent journey resulting in one chat reply.

Intelligence nodes may define business logic, can invoke the LLM one or more times, or update state. Intelligence nodes may implement two basic functions: a processor function that may receive the current state during the graph execution, invoke the LLM, change the state, and return a new state, and a transition function that may return the name of the path that may lead to the next node that needs to be executed. The LLM may be invoked to determine which path to take, and then the graph cyclically transitions back to the intelligence node where the LLM can make inferences based on the state and its associated context. Intelligence nodes may provide steps in a process in which the AI agent may make decisions, observations, and update state; while execution nodes may provide steps in a process where the AI agent may interact with other components of the AHAMS.

The LLMs may be used for decision-making, generating chat messages, and selectively invoking customized software routines. The LLMs may be invoked multiple times to complete specific tasks for other components of AHAMS. The LLMs can detect when software functions may be called and then respond to inputs, allowing the AI agent to select execution tools that may be used to complete tasks. In API calls, functions can be described, and the LLM can intelligently output a JSON object containing arguments to call these functions. When the LLM responds with an intent to use execution tools, the execution tools may be implemented within execution nodes. Execution tools may include checking availability of slots for appointments, scheduling appointments, and then modifying or canceling scheduled appointments dynamically.

The autonomous routing server may be used for classifying content of the user request and routing the message to relevant nodes. The autonomous routing server may also be used to plan an appointment that may involve generating a treatment plan for a user based on the availability of resources and other associated information. The autonomous routing server may include two intelligence nodes and an execution node in a graph. Intelligence node 1 may be a message routing/classification node that may classify chat messages into categories such as requested actions, supported actions and unsupported actions. If the LLM determines actions, it can return the path to an intelligence node 2 that schedules appointments of the treatment plan. If no actions are taken or the requested actions are invalid, it may return the path to the chat message synthesizer.

In some aspects, the intelligence node 2 that plans and schedules appointments of the treatment plan may interpret user messages and update the state with a list of appointments that may need to be created, modified, or canceled. The intelligence node 2 may transition to the execution node to identify suitable appointment times for subjects. If planning cannot be completed due to an incorrect or missing information, it may return the path to the chat message synthesizer. If planning can be completed and significant information is collected, it may return the name of one or more paths leading to different nodes in the autonomous scheduling server. For complex requests, the intelligence node 2 may request a follow-up to confirm subsequent scheduling actions. The execution node may keep checking whether subjects are available for appointments with providers by sending messages through external messaging systems.

The autonomous scheduling server may be used to create, cancel, and modify appointments by using different execution tools. The intelligence and execution nodes of the autonomous routing and scheduling server may function in parallel to each other for enhancing the speed of managing and processing appointments. The autonomous scheduling server may be triggered by the autonomous routing server once a treatment plan of subject is created. The autonomous scheduling server may consist of four intelligence nodes and an execution node. The first intelligence node may create appointments using information from the state, triggering a transition to the execution node. The results of an execution tool in an instance are saved in the state. The second intelligence node may modify existing appointments using state information, triggering a transition to the execution node. The third intelligence node may cancel existing appointments using state information, triggering a transition to the execution node. The fourth intelligence node may aggregate the results and may function as an arbiter, which may execute once actions the appointment creation, modification, or cancellation intelligence nodes are completed.

In one embodiment, the autonomous scheduling server may have only one transition to the chat message synthesizer. The node sweeps over the results of each appointment action, adding an indication of a follow-up message if any action was unsuccessful. If all appointment action outcomes are successful, no follow-up information may be provided. The execution node may execute changes to an underlying healthcare scheduling system through the healthcare systems integration layer. The healthcare systems integration layer may provide access to various tools for creating new appointments, modifying existing appointments, or canceling existing appointments.

In yet another embodiment, the chat message synthesizer is a single intelligence node in the AI agent graph that may understand changes in the state once the graph instance is executed and then subsequently may generate a concise chat reply for a user. If a follow-up information is present, the chat message synthesizer may prompt the user for relevant information. If no follow-up information is present, the chat message synthesizer may produce a user-friendly summary of all actions done during the time a graph's instance was executed.

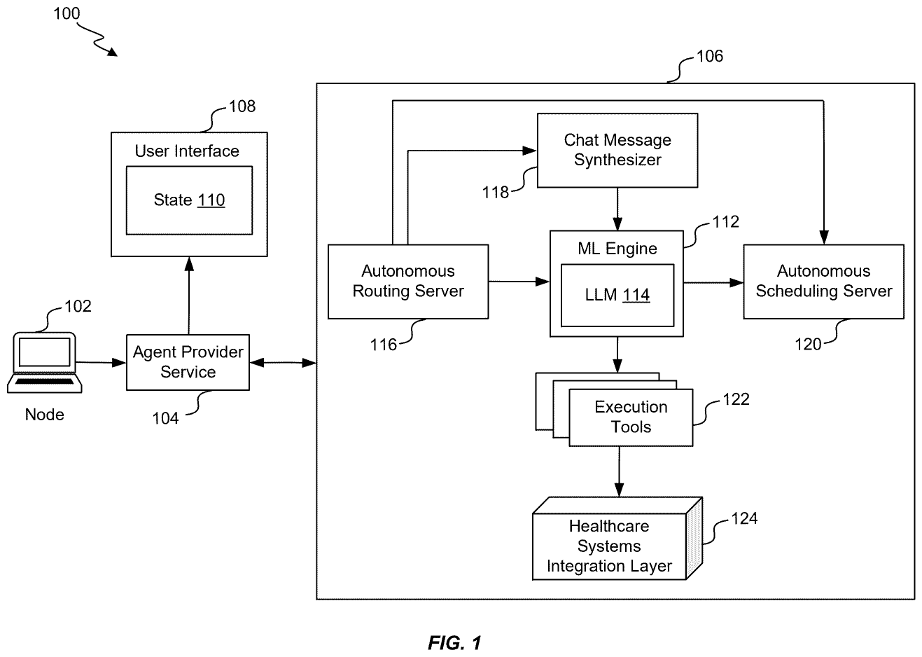

FIG. 1 illustrates an exemplary block diagram 100 of an autonomous healthcare appointment management system (AHAMS) 106 along with an agent provider service 104 in accordance with some aspects of the present disclosure. The exemplary block diagram 100 may include a node 102, the agent provider service 104, and a user interface 108 including a state 110. The state 110 may provide a chatbot-style data exchange or interaction between the user and the AI agent, at the user interface 108. The state 110 may provide chat message history containing both user and AI-generated messages.

The AHAMS 106 may be a software solution to streamline the process of scheduling, rescheduling, or canceling subjects' appointments with providers. The AHAMS 106 may use technologies such as artificial intelligence (AI) and machine-learning to manage subjects' appointments and send automated reminders to optimize the allocation of healthcare resources. By integrating the AHAMS 106 with electronic health records (EHR) and other healthcare databases, the AHAMS 106 may keep an updated user's medical information that is accessible to both users and healthcare providers. This not only reduces administrative burdens but also enhances users' satisfaction by minimizing waiting times and improving the quality of overall care due to a better coordination.

Additionally, the AHAMS 106 can analyze appointment patterns and then may predict future appointment trends, permitting healthcare facilities to better manage their capacity, resources, and service delivery. In one embodiment, the AHAMS 106 can be an AI agent interacting with the agent provider service 104. The AI agent may be a software program that can autonomously perform tasks on behalf of a user or a system that is used by the user. The AI agent may be responsible for scheduling healthcare appointments and understanding treatment plans by interacting with users in a chatbot-style process. In this disclosure, the terms “AHAMS” and “AI agent” are used interchangeably.

The node 102 may be a user (i.e., a subject/patient, an administrator, etc.) on a user device (i.e., a laptop, a cell phone, a computer, etc.) accessing the AHAMS 106 via the agent provider service 104. In this application, the term “a user” or “a subject” refers to the node 102. The agent provider service 104 may refer to the infrastructure and support system that can host and maintain the AI agent or the AHAMS 106. The agent provider service 104 may provide resources and environment for the AI agent to function correctly, maintain state and context between interactions, and then execute associated tasks efficiently in an effective manner. The agent provider service 104 may be communicatively coupled with the user interface 108 of the user device. The user interface 108 may include the state 110 that may imply a current data exchange between the node 102 and the AHAMS 106, both interacting in a natural language in a chatbot-style process. The AHAMS 106 may include a machine-learning (ML) engine 112 that may further include a large language model (LLM) 114, an autonomous routing server 116, a chat message synthesizer 118, and an autonomous scheduling server 120. The AHAMS 106 may further include one or more execution tools 122 that are integrated with a healthcare systems integration layer 124.

The ML engine 112 may utilize algorithms to analyze large amounts of big healthcare data of subjects. The ML engine 112 may include the LLM 114, which may be designed to understand and generate response in a natural language text. The LLM 114 may help in interpreting user input, understanding context of user requests, and providing relevant responses. The ML engine 112 continuously learns from interactions, improving its accuracy and effectiveness over a time. The LLM 114 may be capable of selectively invoking custom software routines, referred to as the execution tools 122. The LLM 114 may leverage its sophisticated natural language processing capabilities to understand and respond to complex queries by users or providers. The LLM 114 can be invoked a fixed number of times to complete specific tasks for other components (or modules) within the AHAMS 106. By limiting the number of times these LLMs can be invoked, the AHAMS 106 ensures efficient use of resources while maintaining a high performance. The LLM 114 may have the ability to manage multiple tasks simultaneously and provide accurate responses.

The LLMs can be fine-tuned to detect when one or more software functions or the execution tools 122 need to be called, and the LLMs may provide a response with appropriate inputs for these functions. This fine-tuning is not a requirement for function calling but serves as a convenience to streamline the scheduling process for appointments. A user might decide to use a tool to complete a task, the AI agent can determine when to use the execution tool 122 to assist in completing the task initiated by the user. In an API call to the LLM 114, functions can be described, and the LLM 114 may intelligently choose to output its response in a JSON object containing the necessary arguments to call these functions. When the LLM 114 responds with the intent to use tools, the execution tools 122 are executed, and the results may be provided to the LLM 114 for subsequent inference making steps.

The autonomous routing server 116 may be responsible for directing the flow of information within the AHAMS 106. The autonomous routing server 116 may direct that user requests are routed to the appropriate components of the AHAMS 106 for further processing. For instance, if a user wants to schedule an appointment, the autonomous routing server 116 may direct this request to the autonomous scheduling server 120. The chat message synthesizer 118 may generate coherent and contextually appropriate responses to user requests. The chat message synthesizer 118 may synthesize information from various sources within the AHAMS 106 to provide appropriate and relevant responses to the queries. The chat message synthesizer 118 may generate replies in a natural language at the user interface 108.

The autonomous scheduling server 120 may manage the actual scheduling of appointments. The autonomous scheduling server 120 can process user requests to create, modify, or cancel appointments, ensuring that the changes are also accurately reflected in the AHAMS 106. The autonomous scheduling server 120 may use advanced optimization algorithms to optimize appointment slots, reducing waiting times and efficiently doing resource utilization. The execution tools 122 are utilities or custom software routines that may integrate with the healthcare systems integration layer 124. The execution tools 122 may execute the necessary actions to update the healthcare information management system based on user requests. The execution tools 122 may retrieve additional information from the user or fill in the information using the AI agent when creating a new appointment, modifying an existing one, or canceling an appointment.

The healthcare systems integration layer 124 may provide the interface between the AHAMS 106 and the underlying healthcare scheduling and appointment systems. The healthcare systems integration layer 124 may provide access to the underlying healthcare scheduling and appointment system, enabling management and updates of appointments. The healthcare systems integration layer 124 may use direct interfaces to create, modify, or cancel appointments within the healthcare information management system by doing a seamless integration and management of subjects'appointments.

In one aspect, the agent provider service 104 may offer a session-based interface that allows users to interact with the AI agent through conversational input. The agent provider service 104 may function as a chatbot, facilitating communication between the user and the AI agent in a natural language. The agent provider service 104 can maintain session information, known as the state 110, which includes chat message history containing both user and AI-generated messages. The state 110 may provide a chatbot-style data exchange or interaction between the user and the AI agent.

The agent provider service 104 may also manage feedback metadata, such as follow-up flags indicating that the next user message is a follow-up message containing supplemental information for the autonomous routing server 116. The follow-up requests, which are messages generated by the AI agent, may ask for additional information from the user. Additionally, the agent provider service 104 can also manage changes in the list of appointments of the treatment plan and manage the lists that are used to track the appointment-related tasks. For example, these lists may include an appointment creation list containing information about proposed appointments that may need to be created, an appointment modification list containing appointments that may need to be modified, and an appointment cancellation list containing appointments that may need to be cancelled. Individual items in these lists are populated with successful outcomes or error messages, if any, after the autonomous scheduling server 120 completes its tasks, and this information is later used by the chat message synthesizer 118.

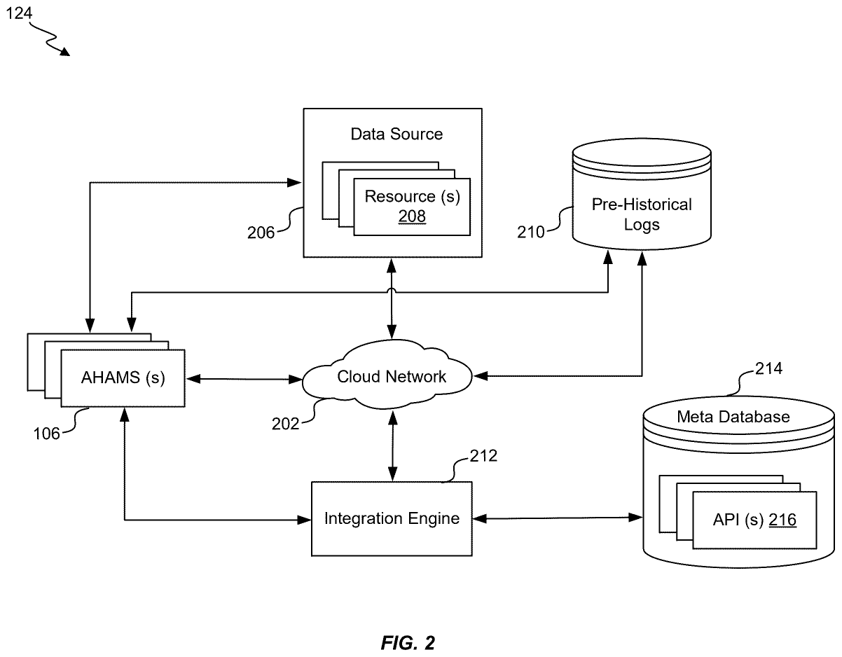

FIG. 2 illustrates a block diagram of a healthcare systems integration layer 124 at a cloud network 202 in accordance with some aspects of the present disclosure. The healthcare systems integration layer 124 may refer to a model that enables different healthcare systems, applications, and devices to communicate and work together in a seamless fashion. The healthcare systems integration layer 124 may include the AHAMS(s) 106, a data source 206 containing resources 208 of the hospitals, pre-historical logs 210, a meta database 214 including application programming interfaces (APIs) 216, and an integration engine 212, all are communicating over the cloud network 202.

The healthcare systems integration layer 124 may use interoperability standards like health level 7 (HL7), fast healthcare interoperability resources (FHIR), and digital imaging and communications in medicine (DICOM) to exchange data between different systems. The data source 206 is a repository that may give access to the AHAMS 106 to the resources (i.e., reports, calendars, patient/staff schedules, policies, electronic health records (EHR), etc.) of the hospitals. This access and details of the resources 208 may allow the AHAMS 106 to route and schedule the user request in a seamless fashion according to service needs of the user. The pre-historical logs 210 may be used by the AHAMS 106 to build a context specific to a user and understand the semantics of the content of the request while routing the request. The pre-historical logs 210 may include past encounters with a user, with healthcare providers, and a user's preferences that may further be used to generate AI-based chat responses at the user interface 108. The execution tools 122 of the AHAMS 106 may also use the pre-historical logs 210 to fill in any additional information while scheduling an appointment for the user.

The AHAMS 106 may receive paths to the different resources of the hospitals using the APIs 216 from the meta database 214. The meta database 214 may store the information (i.e., APIs, healthcare plans, staff, medical facilities, etc.) corresponding to the hospitals which are accessing the AHAMS 106 at the cloud network 202. The meta database 214 may return the APIs 216 of the available resources for scheduling the user request for an appointment. The APIs 216 may return the path to different nodes of the AI agent or the AHAMS 106 to execute the request processing. The integration engine 212 may manage data flow, handle requests, and allow data exchange between different components working at the cloud network 202. The integration engine 212 may use different tools to manage data exchange between different modules. Examples of such tools may include Mirth Connect and Cloverleaf that can help in transforming and routing user requests between different AHAMS working either inside or outside of the cloud network 202.

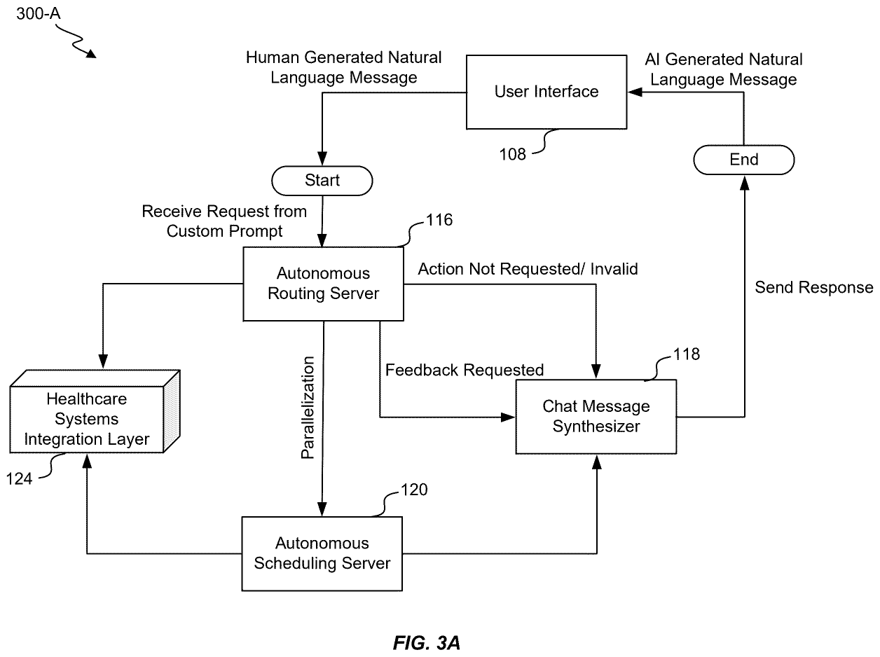

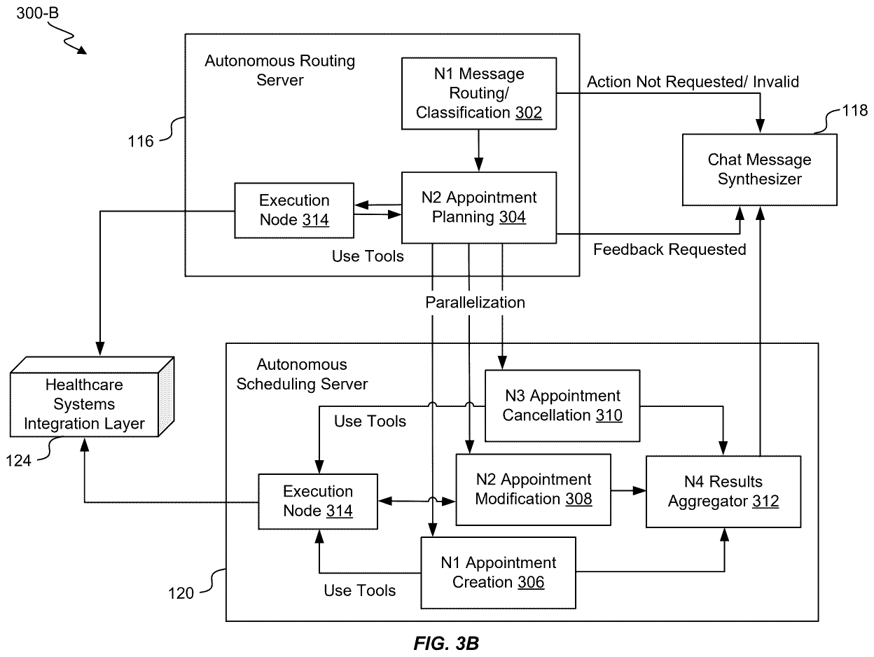

FIG. 3A illustrates a sequence diagram 300-A for routing and scheduling the request via the AHAMS 106. The request routing and scheduling process may be divided into the following aspects: advanced reasoning and function-calling capabilities leveraging the LLM 114, the autonomous routing server 116, the autonomous scheduling server 120, the chat message synthesizer 118, the agent provider service 104, and the healthcare systems integration layer 124. The agent provider service 104 may provide a session at the user interface 108 to give conversational access to the AI agent. The agent provider service 104 may facilitate conversation with the user at the node 102, acting as a chatbot agent to allow interactions with the user in a natural language data. The agent provider service 104 may also maintain a session information at the user interface 108, called the state 110. The state 110 may include the following information: chat message history containing both user and AI generated messages, feedback metadata, appointment change lists, etc.

The user interface 108 may receive a user generated message in a natural language at the state 110. The user interface 108 forwards the message to the autonomous routing server 116. In a first step of the request routing and scheduling, the AHAMS 106 may receive request in a natural language along with the custom prompt in the state 110. The autonomous routing server 116 may extract information from the request of the user to understand semantics of the content and the context of the request and then plan request's routing accordingly.

The autonomous routing server 116 of the AHAMS 106 coordinates with the healthcare systems integration layer 124 to collect data and APIs from across the different health providers in a network. The autonomous routing server 116 may use different nodes of the AI agent to plan and route the requests of the user. The nodes of the autonomous routing server 116 may include two intelligence nodes and one execution node, the details of which will be given later in this application. The intelligence nodes may define business logic and can change the state 110 at the user interface 108 while the execution nodes may provide customized software routines or the execution tools 122 to interact with other components or the user. The autonomous routing server 116 may generate a treatment plan for the user based on its request and preferences. The treatment plan may refer to a medical plan that may include structured data indicating appointment scheduled, managing, and tracking appointments of users with providers. The treatment plan may include prioritization protocols to ensure that urgent cases are scheduled promptly, yet routine appointments are efficiently planned by optimizing the provider's schedule.

The autonomous scheduling server 120 may be triggered by the autonomous routing server 116 once the treatment plan is supplied. The autonomous scheduling server 120 may include four intelligence nodes and one execution node which will be described later in the draft. The appointment creation, modification, and cancellation intelligence nodes may all process their tasks at the same time in parallel to increase overall processing speeds for large requests. The autonomous scheduling server 120 may retrieve a list of available resources and free slots from the data source 206 and verify the availability of the resources 208 within a time frame. The autonomous scheduling server 120 may then request the APIs 216 and schedule appointments for the users according to corresponding treatment plans at the available slots and resources.

The chat message synthesizer 118 may be represented as a single intelligence node at the AI agent and may always be the last node to be executed while processing the request. The chat message synthesizer 118 may understand the changes to the state 110 over the lifetime of one request processing instance and generate a brief chat reply at the user interface 108 for the user. If follow-up information is present in the state 110, then the chat message synthesizer 118 may formulate a prompt for the user along with requesting any relevant information. If no follow-up information is present, then the chat message synthesizer 118 may produce a chat reply which summarizes in a user-friendly manner everything that was done during the lifetime of one request processing instance. The chat message synthesizer 118 may send the response that may be displayed in a natural language at the user interface 108.

FIG. 3B illustrates a block diagram 300-B for request routing and scheduling using one or more LLM nodes in accordance with some aspects of the present disclosure. The autonomous routing server 116 includes two intelligence nodes, namely an N1 302 for message routing/classification and an N2 304 for appointment planning, and an execution node 314. The N1 302 may be executed when the human chat is combined with the custom prompt and sent to the LLM 114. The LLM 114 classifies the request into two categories: action requested or action not requested/invalid. The action requested category may include one or more scheduling-related actions that may need to be completed. The scheduling actions may include finding availability of resources, and creating, modifying, or cancelling appointments. The invalid category may include the requests in which no action is requested, or an unsupported action is requested.

If the LLM 114 determines that there are actions to take, the transition function of the N1 302 may provide the name of the path leading to the next intelligence node i.e., the N2 304. If the LLM 114 determines that there are no actions to take or the requested actions are invalid, the transition function of the N1 302 may provide the name of the path leading to the chat message synthesizer 118. The N1 302 makes no changes to the state 110. If a follow-up information exists in the state 110, then the user's message is matched to see if the LLM's request for supplemental information is fulfilled. If so, the supplemental information is saved in the state 110 for consumption of appointment planning node (i.e., the N2 304).

The N2 304 is the second intelligence node that may be used for appointment planning. The N2 304 may use the LLM 114 to interpret the user message and update the state 110 partially or completely with a list of appointments to create, modify, or cancel, given in a natural language input. The N2 304 may transition to the execution node 314 iteratively to identify suitable appointment times for one or more users. If the planning of appointments cannot be completed due to incorrect or missing information, the transition function of the N2 304 will return the name of the path leading to the chat message synthesizer 118. If the planning of appointments is complete and a significant amount of information is collected, then the transition function of the N2 304 will return the name of one or more paths, all of which lead to various nodes in the autonomous scheduling server 120. If supplemental information is available in the state 110 because of a follow-up, this may also be provided in a custom prompt to the LLM 114 to auto-complete the missing data during the planning phase. For complex requests, the N2 304 shall request a follow-up to confirm scheduling actions.

The autonomous scheduling server 120 may include four intelligence nodes, namely an N1 306 for an appointment creation, an N2 308 for an appointment modification, an N3 310 for an appointment cancellation, and an N4 312 as results aggregator, while it also has one execution node 314. The N1 306 may rely on information in the state 110 to create appointments. All necessary information is present in the state 110 to take actions to create appointments. For an appointment to create, the LLM 114 may identify the need to use the execution tool 122 to create a new appointment and will cause a transition to the execution node 314. Once the execution tool 122 is used, it may lead to a successful outcome or unsuccessful outcome. The results of each execution tool use-instance are saved in the state 110.

The N2 308 may also rely on information in the state 110 to modify existing appointments. All necessary information is present in the state 110 to take action to modify appointments. For an appointment to modify, the LLM 114 identifies the need to use the execution tool 122 to modify an existing appointment and will cause a transition to the execution node 314. Once the execution tool 122 is used, it may lead to a successful outcome or unsuccessful outcome. Again, the results of each execution tool instance are saved in the state 110. In a similar way, the N3 310 may also rely on information in the state 110 to cancel existing appointments. All required information is present in the state 110 to take action to cancel appointments. For an appointment to cancel, the LLM 114 identifies the need to use the execution tool 122 to cancel an existing appointment and will cause a transition to the execution node 314. Once the execution tool 122 is used, it may lead to a successful outcome or unsuccessful outcome. Again, the results of each execution tool instance are saved in the state 110.

The N4 312 is the last intelligence node of the autonomous scheduling server 120 that may be used as a results aggregator or arbiter. The N4 312 may be executed only when the appointment creation, appointment modification, or appointment cancellation intelligence nodes are complete regardless of the outcomes (i.e., successful, or not successful). The N4 312 can only have one transition to the chat message synthesizer 118. The N4 312 may sweep over the results of each appointment action (creation, modification, or cancellation). If an appointment's action is unsuccessful, then the N4 312 will add to the state 110 an indication that a follow-up message is requested and provide relevant information to the chat message synthesizer 118. If all appointment action outcomes are successful, then no follow-up information is provided to the chat message synthesizer 118 via the state 110.

The execution node 314 may provide the capabilities for the appointment creation, appointment modification, or appointment cancellation intelligence nodes to execute changes to the underlying healthcare scheduling system through the healthcare systems integration layer 124.

The execution node 314 may provide access to a set of execution tools 122 to create new appointments, modify existing appointments, or cancel existing appointments. The autonomous scheduling server 120 may be triggered by the autonomous routing server 116 once the treatment plan for a user is created. The appointment creation, appointment modification, and appointment cancellation intelligence nodes may all process their tasks at the same time in parallel to increase overall processing speeds for large requests.

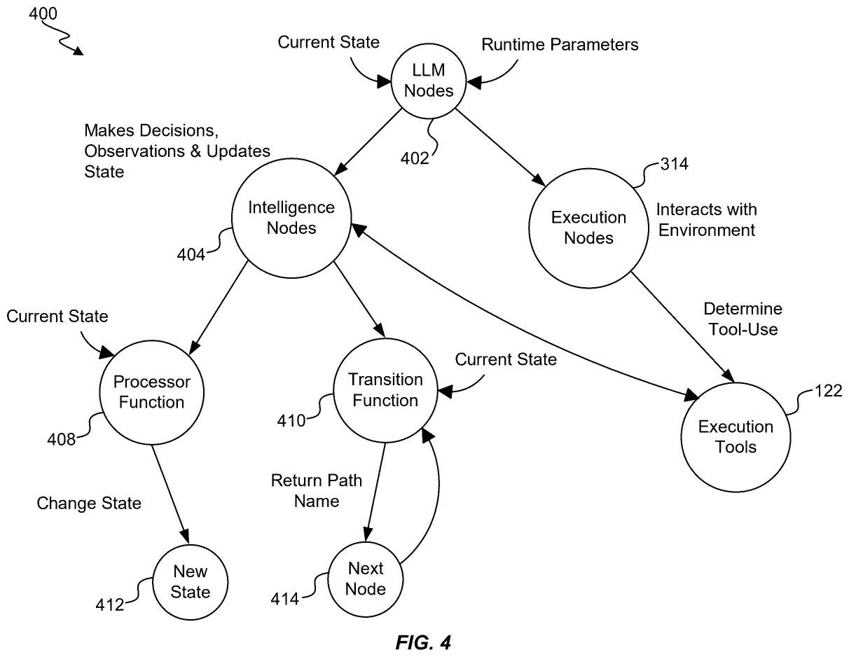

FIG. 4 shows an AI agent graph 400 for transitioning between one or more LLM nodes 402 in accordance with some aspects of the present disclosure. The AHAMS 106 may be modeled as an AI agent along with the agent provider service 104 to provide the AI agent with different services and also maintain the state 110 and context between graph executions. The AI agent may use a static directed cyclic graph. The AI agent may plan for scheduling of the request and dynamically change the schedule of the request based on different parameters such as subject availability, resource availability, change in wait period, medical emergencies, etc. The input to the cyclic graph may be the current state and the runtime parameters (i.e., custom prompt, user credentials, etc.) that are provided as input to the LLM nodes 402. The LLM nodes 402 in this graph may take one of two types (i.e., intelligence nodes 404 or execution nodes 406). The AI agent graph 400 is executable, and one execution of the AI agent graph 400 may indicate one AI agent journey resulting in one chat reply. The two inputs to the LLM nodes 402 are the states which may contain the chat message history and other data, and runtime parameters. The only output from the AI agent graph 400 is a new state including an AI generated chat response, and errors from executing the graph which may include LLM invocations and the execution tool-use.

The intelligence nodes 404 may define a business logic, can invoke the LLM 114 one or more times, or update the state 110. The intelligence nodes 404 may implement two basic functions: a processor function 408 and a transition function 410. The processor function 408 may refer to an implementation that can receive the current state in the graph execution, invoke the LLM 114, mutate the state 110, and generate a new state 412. The transition function 410 may refer to an implementation which can receive the current state and return the name of the path leading to a next node 414 to execute. The LLM 114 may be invoked to determine which path to take. The transition function 410 may also return different path names, in which case the next nodes 414 are executed in parallel.

The execution nodes 406 may provide access to the execution tools 122, also known as custom software routines. The AI agent may determine whether tool-use is necessary before transitioning to another intelligence node and if the answer is yes then it shall transition to the execution node 314 where tools/custom software routines are invoked. The outcomes of the execution tools 122 may be made available and the graph cyclically transitions back to the intelligence node 404 where the LLM 114 can make its inferences based on input prompts. When the LLM 114 is invoked within an intelligence node 404, the LLM 114 can be informed of which execution tools it might use. The execution tools 122 may be used to check the availability of resources over a specific period (look up existing schedule/slot/appointments, etc. for an identified healthcare provider) to create a new appointment, or to modify/cancel an existing appointment. Note that the intelligence nodes 404 are the steps in the scheduling process, and the AI agent may make decisions, observations, and update the state of users'appointments. In comparison, the execution nodes 406 are the steps in the scheduling process where the AI agent may interact with other components of the AHAMS 106.

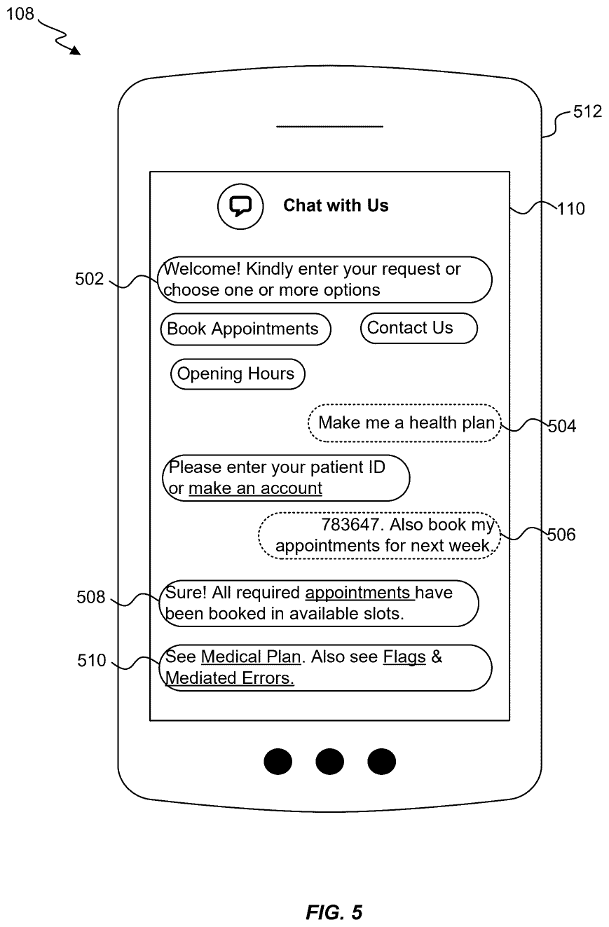

FIG. 5 shows the user interface 108 illustrating the state 110 on a user device 512 according to some aspects of the present disclosure. The user interface 108 shows a chatbot-interaction 502 with the user at the node 102. The agent provider service 104 may facilitate conversation with the user at the node 102, acting as a chatbot agent with the user in a natural language data 506 with the user. The agent provider service 104 may also maintain session information at the user interface 108, called the state 110. The state 110 may include the following information: chat message history containing both user and AI generated messages, feedback metadata, appointment change lists, etc. The user interface 108 may receive a user generated message in natural language at the state 110. The user interface 108 may forward a request 504 to the autonomous routing server 116.

The chat message synthesizer 118 may also ask for a follow up information from the user. If no follow-up information is required, then the chat message synthesizer 118 may generate a chat reply with a list of appointments 508 which fulfills the user request. The list of appointments 508 may be displayed at the state 110. The chat message synthesizer 118 may provide the user with a treatment plan 510 along with a list of flags and a log of errors that are mediated by the LLM 114. The list of flags and mediated errors may indicate the supplemental information provided by the LLM 114 or corrections in the treatment plan 510. The chat message synthesizer118 may also summarize the response in a user-friendly manner that may include everything that may be done during the lifetime of the graph's execution instance.

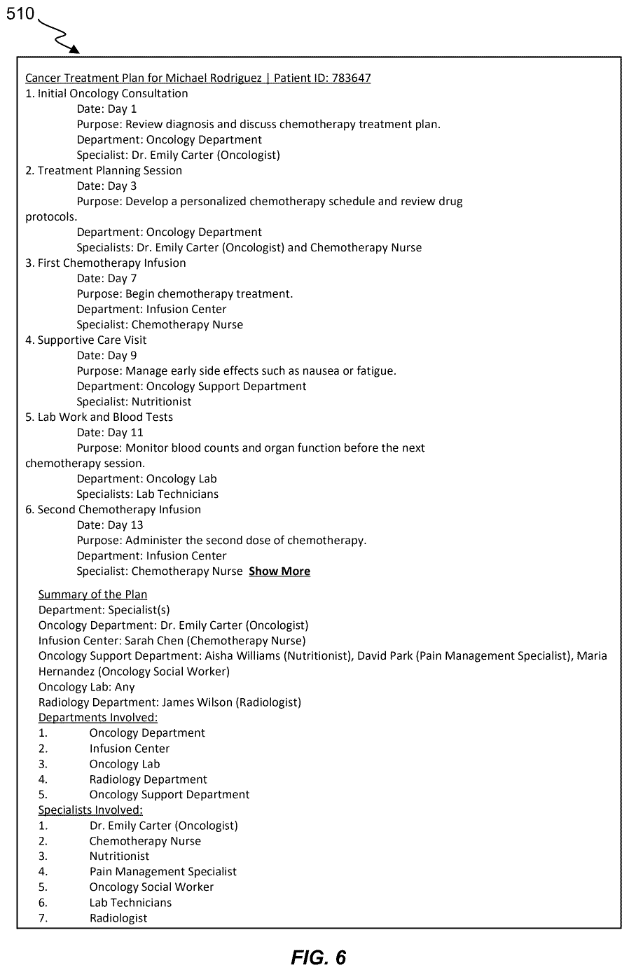

FIG. 6 shows a treatment plan 510 developed by the AHAMS 106 for a user using the LLM 114 of the ML engine 112. The cancer treatment plan for a user with ID 783647 is scheduled using the AI agent to ensure there are no clashes or overlaps in appointments. The AHAMS 106 may analyze the availability of various departments and specialist physicians that may be involved in administering this treatment. By considering factors such as the duration of each appointment, travel time between departments, and the subject's overall health state, the AHAMS 106 may ensure that appointments are spaced out appropriately.

If an overlap is identified, the AHAMS 106 can automatically adjust the schedule, rescheduling appointments to the next available slot that fits within the treatment timeline. This helps in allocating resources such as medical staff, equipment, and treatment rooms. Each appointment is adequately staffed and equipped, minimizing waiting times and enhancing the quality of care.

The AHAMS 106 may also consider the user's specific needs according to the treatment plan 510 to avoid the unnecessary delays. This may include scheduling supportive care visits and lab tests at suitable times to better manage side effects and monitor the health state of the user efficiently and effectively. The AHAMS 106 is a live system that may monitor the treatment schedule continuously in real-time and then can also adjust in real-time. For example, if a user experiences severe side effects, the AHAMS 106 can prioritize supportive care visits and reschedule other appointments accordingly. In one embodiment, the AHAMS 106 can also use additional LLMs or fuzzy logic controls to overcome the human-error or eliminate behavioral delays during the encounters at a healthcare facility. This may reduce unnecessary delays for the users, enabling them to effectively plan their treatment administration and other activities accordingly.

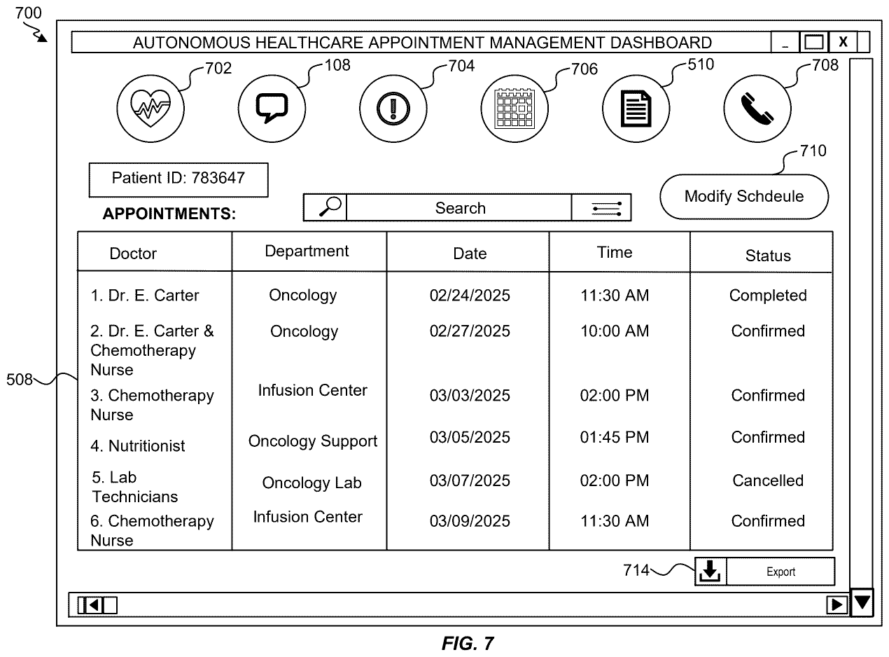

FIG. 7 illustrates an autonomous healthcare appointment management dashboard 700 on a user device showing a list of appointments 508 with associated details. The autonomous healthcare appointment management dashboard 700 may be accessed by the user to manage appointment requests and track their status. The autonomous healthcare appointment management dashboard 700 may also provide additional information such as medical care 702, chat with a medical bot at the user interface 108, a list of flags 704, reminders in the calendar 706, the treatment plan 510, and a contact 708 at the hospital/health providers. The autonomous healthcare appointment management dashboard 700 may also allow the user to modify their schedule 710 according to user's preferences and may save that information to make subsequent appointments. The list of appointments 508 may include the name of a doctor/medical staff, the name of departments of one or more hospitals working on the case or having the treatment facility, the date and time of the appointments, and the status of the appointments. The status of the appointment may indicate whether the appointment has been completed, confirmed/scheduled, or cancelled. The user may request modifications to the schedule or reschedule appointment using the autonomous healthcare appointment management dashboard 700. The user may also export the details 714 displayed at the autonomous healthcare appointment management dashboard 700 to an external entity.

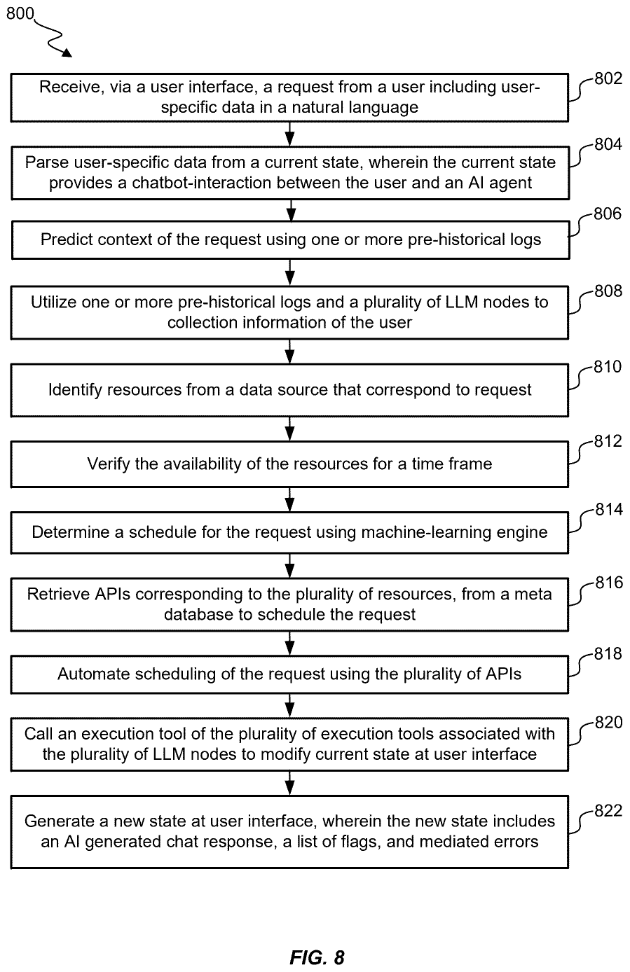

FIG. 8 illustrates a flowchart 800 of a method for automating healthcare appointments and managing user requests according to some aspects of the present disclosure. The disclosed method in the flowchart 800 may dictate an AI agent to automate natural language interactions with users to autonomously coordinate and schedule appointments. At block 802, a request from a user including user-specific data in a natural language may be received via the user interface 108. At block 804, user-specific data may be parsed from the current state of the user interface 108 and analyzed for further processing. The current state may indicate a chatbot-interaction between the user and the AI agent at a given instant of time. At block 806, the natural language request may be sent to the AHAMS 106. The AHAMS 106 may predict the context of the request using the pre-historical logs 210. At block 808, the pre-historical logs 210 and the LLM nodes 402 may be utilized to collect information about the user. At block 810, based on the context and information of the user, the resources 208 may be identified from the data source 206 that corresponds to the request. These resources may include reports, calendars, patient/staff schedules, policies, EHR, and the like, of the healthcare facilities (i.e., hospitals, clinics, laboratories, etc.).

At block 812, availability of the resources 208 in a time slot may be verified. At block 814, the AHAMS 106 may determine a schedule for the user request using the ML engine 112 based on the verification of resource availability. At block 816, the APIs 216 corresponding to the resources 208 may be retrieved from the meta database 214, to schedule the request. At block 818, the scheduling of the request may be automated using the APIs 216. At block 820, the intelligence nodes of the AI agent may call the execution tools 122 to modify the current state of the user interface 108. Finally at block 822, a new state may be generated on the user interface 108 by the chat message synthesizer 118. The new state may include an AI generated chat response, a list of flags, and mediated errors.

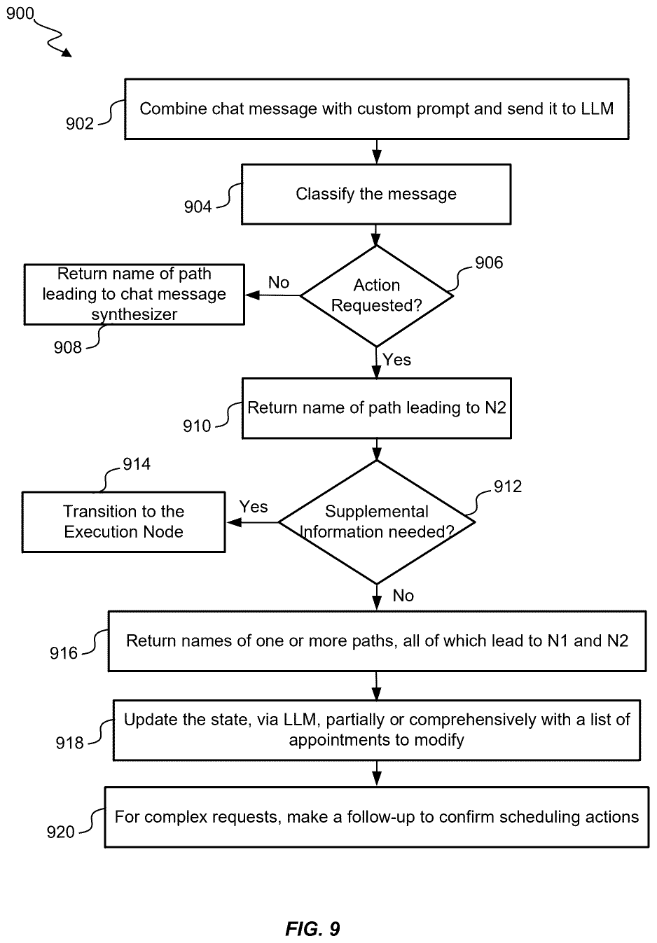

FIG. 9 illustrates an exemplary flowchart 900 for planning and creating an appointment by the AHAMS 106 as an embodiment of the present disclosure. The exemplary flowchart 900 may depict the appointment planning and creation process conducted by the LLM nodes 402 of the AI agent. At block 902, the AHAMS 106 may combine a chat message with a custom prompt at a current state and send it to the LLM 114. At block 904, the N1 302 may classify the content of the message/request. At block 906, the AHAMS 106 checks whether any action is requested in the message or not? If an action is not requested or an unsupported action is requested, then the transition function of this intelligence node may return the name of path leading to the chat message synthesizer 118 at block 908.

On the other hand, if an action is requested by the user at block 906, then the N1 302 may return the name of the path leading to the second intelligence node (i.e., N2 304) at block 910. At block 912, the N1 302 may analyze whether supplemental information is needed to fulfill the request. If additional information is needed, then N1 302 may transition to the execution node 314 to get access to the execution tools 122 at block 914. On the other hand, if no further information is needed, then the supplemental information may be saved in the state 110 and names of one or more paths may be returned at block 916, all of which may lead to the N1 302 and N2 304.

At block 918, the appointment planning node (i.e., N2 304) may use the LLM 114 to interpret the request and update the state 110 state partially or completely with a list of appointments to create, modify, and cancel, given in a natural language input. At block 918, the N2 304 may transition to the execution node 314 repeatedly to identify suitable appointment times for various users. The N2 304 may invoke the autonomous scheduling server 120 to automate the request scheduling process. Finally at block 920, the autonomous routing server 116 may make a follow-up request to confirm subsequent scheduling actions (in case of complex requests).

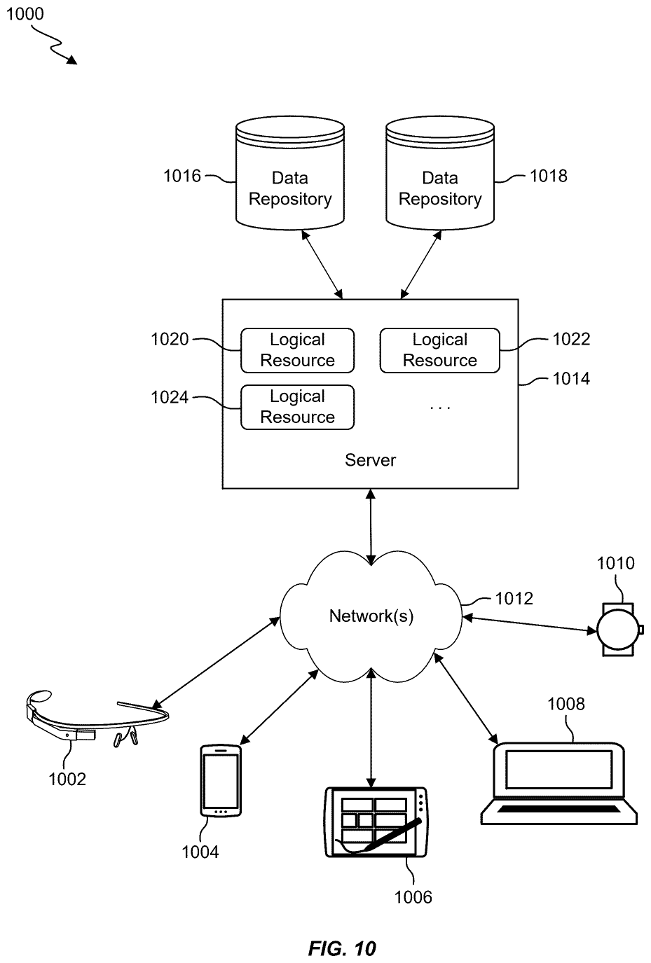

FIG. 10 depicts a simplified diagram of a distributed system 1000 for implementing an embodiment. In the illustrated embodiment, distributed system 1000 includes one or more client computing devices 1002, 1004, 1006, 1008, 1010, coupled to a server 1014 via one or more communication networks. The client computing devices 1002, 1004, 1006, 1008, 1010, may be configured to execute one or more applications. In various aspects, server 1014 may be adapted to run one or more services or software applications that enable techniques for the secure communication network. In certain aspects, server 1014 may also provide other services or software applications that can include non-virtual and virtual environments. In some aspects, these services may be offered as web-based or cloud services, such as under a Software as a Service (SaaS) model to the users of client computing devices 1002, 1004, 1006, 1008, 1010. Users operating the client computing devices 1002, 1004, 1006, 1008, 1010, may in turn utilize one or more client applications to interact with server 1014 to utilize the services provided by these components.

In the configuration depicted in FIG. 10, server 1014 may include one or more components 1020, 1022 and 1024 that implement the functions performed by server 1014. These components may include software components that may be executed by one or more processors, hardware components, or combinations thereof. It should be appreciated that various system configurations are possible, which may be different from distributed system 1000. The embodiment shown in FIG. 10 is thus one example of a distributed system for implementing an embodiment system and is not intended to be limiting.

Users may use the client computing devices 1002, 1004, 1006, 1008, 1010, for techniques in accordance with the teachings of this disclosure. A client device may provide an interface that enables a user of the client device to interact with the client device. The client device may also output information to the user via this interface. Although FIG. 10 depicts only five client computing devices, any number of client computing devices may be supported.

The client devices may include various types of computing systems such as smart phones or other portable handheld devices, general purpose computers such as personal computers and laptops, workstation computers, personal assistant devices, smart watches, smart glasses, or other wearable devices, equipment firmware, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux® or Linux-like operating systems such as Oracle® Linux and Google Chrome® OS) including various mobile operating systems (e.g., Microsoft Windows Mobile®, iOS®, Windows Phone®, Android®, HarmonyOS®, Tizen®, KaiOS®, Sailfish® OS, Ubuntu® Touch, CalyxOS®). Portable handheld devices may include cellular phones, smartphones, (e.g., an iPhone®), tablets (e.g., iPad®), and the like. Virtual personal assistants such as Amazon® Alexa®, Google® Assistant, Microsoft® Cortana®, Apple® Siri®, and others may be implemented on devices with a microphone and/or camera to receive user or environmental inputs, as well as a speaker and/or display to respond to the inputs.

Wearable devices may include Apple® Watch, Samsung Galaxy® Watch, Meta Quest®, Ray-Ban® Meta® smart glasses, Snap® Spectacles, and other devices. Gaming systems may include various handheld gaming devices, Internet-enabled gaming devices (e.g., a Microsoft Xbox® gaming console with or without a Kinect® gesture input device, Sony PlayStation® system, Nintendo Switch®, and other devices), and the like. The client devices may be capable of executing various applications such as various Internet-related apps, communication applications (e.g., e-mail applications, short message service (SMS) applications) and may use various communication protocols.

Network(s) 1012 may be any type of network familiar to those skilled in the art that can support data communication using any of a variety of available protocols, including without limitation TCP/IP (transmission control protocol/Internet protocol), SNA (systems network architecture), IPX (Internet packet exchange), AppleTalk®, and the like. Merely by way of example, network(s) 1012 can be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Server 1014 may be composed of one or more general purpose computers, specialized server computers (including, by way of example, PC (personal computer) servers, UNIX® servers, LINIX® servers, mid-range servers, mainframe computers, rack-mounted servers, etc.), server farms, server clusters, a Real Application Cluster (RAC), database servers, or any other appropriate arrangement and/or combination. Server 1014 can include one or more virtual machines running virtual operating systems, or other computing architectures involving virtualization such as one or more flexible pools of logical storage devices that can be virtualized to maintain virtual storage devices for the server. In various aspects, server 1014 may be adapted to run one or more services or software applications that provide the functionality described in the foregoing disclosure.

The computing systems in server 1014 may run one or more operating systems including any of those discussed above, as well as any commercially available server operating system. Server 1014 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, SAP®, Amazon®, Sybase®, IBM® (International Business Machines), and the like.

In some implementations, server 1014 may include one or more applications to analyze and consolidate data feeds and/or event updates received from users of client computing devices 1002, 1004, 1006, 1008, 1010. As an example, data feeds and/or event updates may include blog feeds, Threads® feeds, Twitter® feeds, Facebook® updates or real-time updates received from one or more third party information sources and continuous data streams, which may include real-time events related to sensor data applications, financial tickers, network performance measuring tools (e.g., network monitoring and traffic management applications), clickstream analysis tools, automobile traffic monitoring, and the like. Server 1014 may also include one or more applications to display the data feeds and/or real-time events via one or more display devices of client computing devices 1002, 1004, 1006, 1008, 1010.

The distributed system 1000 may also include one or more data repositories 1016, 1018. These data repositories may be used to store data and other information in certain aspects. For example, one or more of the data repositories 1016, 1018 may be used to store information for techniques disclosed herein. Data repositories 1016 and 1018 may reside in a variety of locations. For example, a data repository used by server 1014 may be local to server 1014 or may be remote from server 1014 and in communication with server 1014 via a network-based or dedicated connection. Data repositories 1016 and 1018 may be of several types. In certain aspects, a data repository used by server 1014 may be a database, for example, a relational database, a container database, an Exadata® storage device, or other data storage and retrieval tools such as databases provided by Oracle Corporation® and other vendors. One or more of these databases may be adapted to enable storage, update, and retrieval of data to and from the database in response to structured query language (SQL)-formatted commands.

In certain aspects, one or more data repositories 1016, 1018 may also be used by applications to store application data. The data repositories used by applications may be of several types such as, for example, a key-value store repository, an object store repository, or a general storage repository supported by a file system.

In one embodiment, server 1014 is part of a cloud-based system environment in which diverse services may be offered as cloud services, for a single tenant or for multiple tenants where data, requests, and other information specific to the tenant are kept private from each tenant. In the cloud-based system environment, multiple servers may communicate with each other to perform the work requested by client devices from the same or multiple tenants. The servers communicate on a cloud-side network that is not accessible to the client devices to perform the requested services and keep tenant data confidential from other tenants.

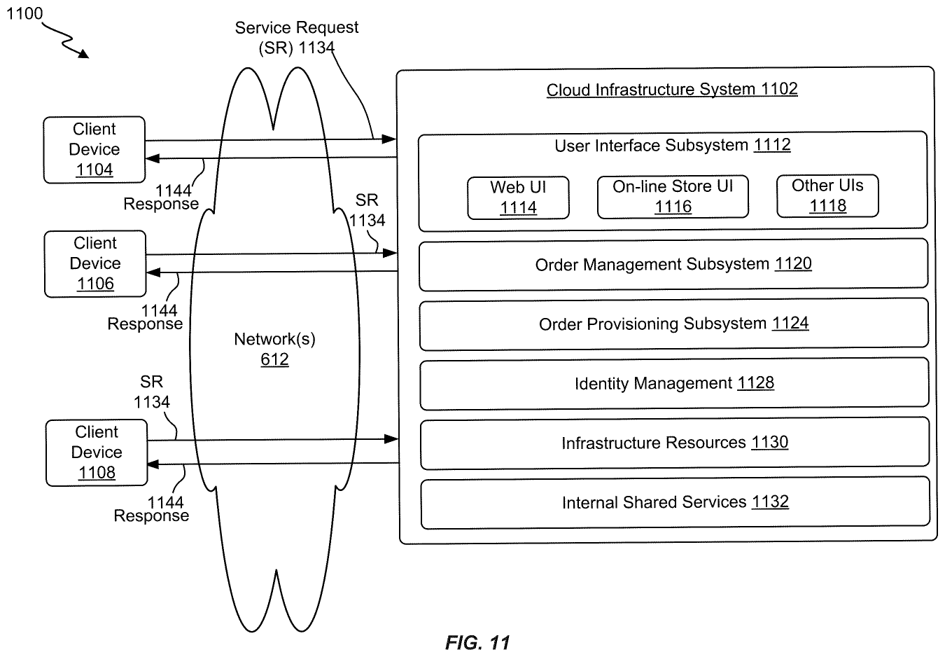

FIG. 11 is a simplified block diagram of a cloud-based system environment 1100 in accordance with certain aspects of various embodiments of the invention. In the embodiment depicted in FIG. 11, cloud infrastructure system 1102 may provide one or more cloud services that may be requested by users using one or more client devices 1104, 1106, and 1108. Cloud infrastructure system 1102 may comprise one or more computers and/or servers that may include those described above for server 1014. The computers in cloud infrastructure system 1102 may be organized as general-purpose computers, specialized server computers, server farms, server clusters, or any other appropriate arrangement and/or combination.

Network(s) 1012 may facilitate communication and exchange of data between the client devices 1104, 1106, and 1108 and cloud infrastructure system 1102. Network(s) 1012 may include one or more networks. The networks may be of the same or several types. Network(s) 1012 may support one or more communication protocols, including wired and/or wireless protocols, for facilitating the communications.

The embodiment depicted in FIG. 11 is only one example of a cloud infrastructure system and is not intended to be limiting. It should be appreciated that, in some other aspects, cloud infrastructure system 1102 may have more or fewer components than those depicted in FIG. 11, may combine two or more components, or may have a different configuration or arrangement of components. For example, although FIG. 11 depicts three client devices, any number of client devices may be supported in alternative aspects.

The term cloud service is used to refer to a service that is made available to users on demand and via a communication network such as the Internet by systems (e.g., cloud infrastructure system 1102) of a service provider. Typically, in a public cloud environment, servers and systems that make up the cloud service provider's system are different from the cloud customer's (“tenant's”) own on-premises servers and systems. The cloud service provider's systems are managed by the cloud service provider. Tenants can thus avail themselves of cloud services provided by a cloud service provider without having to purchase separate licenses, support, or hardware and software resources for the services. For example, a cloud service provider's system may host an application, and a user may, via the network(s) 1012 (e.g., the Internet), order and use the application without the user having to buy infrastructure resources for executing the application. Cloud services are designed to provide easy, scalable access to applications, resources, and services. Several providers offer cloud services. For example, several cloud services are offered by Oracle Corporation®, such as database services, middleware services, application services, and others.

In certain aspects, cloud infrastructure system 1102 may provide one or more cloud services using different models such as a Software as a Service (SaaS) model, a Platform as a Service (PaaS) model, an Infrastructure as a Service (IaaS) model, a Data as a Service (DaaS) model, and others, including hybrid service models. Cloud infrastructure system 1102 may include a suite of databases, middleware, applications, and/or other resources that enable provision of the various cloud services.

A SaaS model enables an application or software to be delivered to a tenant's client device over a communication network like the Internet, as a service, without the tenant having to buy the hardware or software for the underlying application. For example, a SaaS model may be used to provide tenants with access to on-demand applications that are hosted by cloud infrastructure system 1102. Examples of SaaS services provided by Oracle Corporation® include, without limitation, diverse services for human resources/capital management, client relationship management (CRM), enterprise resource planning (ERP), supply chain management (SCM), enterprise performance management (EPM), analytics services, social applications, and others.

An IaaS model is used to provide infrastructure resources (e.g., servers, storage, hardware, and networking resources) to a tenant as a cloud service to provide elastic compute and storage capabilities. Various IaaS services are provided by Oracle Corporation®.

A PaaS model is used to provide, as a service, platform and environment resources that enable tenants to develop, run, and manage applications and services without the tenant having to procure, build, or maintain such resources. Examples of PaaS services provided by Oracle Corporation® include, without limitation, Oracle Database Cloud Service (DBCS), Oracle Java Cloud Service (JCS), data management cloud service, various application development solutions services, and others.

A DaaS model is used to provide data as a service. Datasets may be searched, combined, summarized, and downloaded or placed into use between applications. For example, user profile data may be updated by one application and provided to another application. As another example, summaries of user profile information generated based on a dataset may be used to enrich another dataset.

Cloud services are provided on an on-demand self-service basis, subscription-based, elastically scalable, reliable, universally available, and secure manners. For example, a tenant, via a subscription order, may order one or more services provided by cloud infrastructure system 1102. Cloud infrastructure system 1102 then performs processing to provide the services requested in the tenant's subscription order. Cloud infrastructure system 1102 may be configured to provide one or even multiple cloud services.

Cloud infrastructure system 1102 may provide the cloud services via different deployment models. In a public cloud model, cloud infrastructure system 1102 may be owned by a third-party cloud services provider and the cloud services are offered to any public tenant, where the tenant can be an individual or an enterprise. In certain other aspects, under a private cloud model, cloud infrastructure system 1102 may be operated within an organization (e.g., within an enterprise organization) and services provided to clients that are within the organization. For example, the clients may be various departments or employees or other individuals of departments of an enterprise such as the Human Resources department, the payroll department, etc., or other individuals of the enterprise. In certain other aspects, under a community cloud model, the cloud infrastructure system 1102 and the services provided may be shared by several organizations in a related community. Various other models such as hybrids of the above-mentioned models may also be used.

Client devices 1104, 1106, and 1108 may be of several types (such as the client computing devices 1002, 1004, 1006, and 1008 depicted in FIG. 7) and may be capable of operating one or more client applications. A user may use a client device to interact with cloud infrastructure system 1102, such as to request a service provided by cloud infrastructure system 1102.

In some aspects, the processing performed by cloud infrastructure system 1102 for providing chatbot services may involve big data analysis. This analysis may involve using, analyzing, and manipulating large data sets to detect and visualize various trends, behaviors, relationships, etc. within the data. This analysis may be performed by one or more processors, processing the data in parallel, performing simulations using the data, and the like. For example, big data analysis may be performed by cloud infrastructure system 1102 for determining the intent of an utterance. The data used for this analysis may include structured data (e.g., data stored in a database or structured according to a structured model) and/or unstructured data (e.g., data blobs (binary large objects)).

As depicted in the embodiment in FIG. 11, cloud infrastructure system 1102 may include infrastructure resources 1130 that are utilized for facilitating the provision of various cloud services offered by cloud infrastructure system 1102. Infrastructure resources 1130 may include, for example, processing resources, storage or memory resources, networking resources, and the like.

In certain aspects, to facilitate efficient provisioning of these resources for supporting the various cloud services provided by cloud infrastructure system 1102 for different tenants, the resources may be bundled into sets of resources or resource modules (also referred to as “pods”). Each resource module or pod may comprise a pre-integrated and optimized combination of resources of one or more types. In certain aspects, different pods may be pre-provisioned for several types of cloud services. For example, a first set of pods may be provisioned for a database service, a second set of pods, which may include a different combination of resources than a pod in the first set of pods, may be provisioned for Java service, and the like. For some services, the resources allocated for provisioning the services may be shared between the services.

Cloud infrastructure system 1102 may internally use services 1132 that are shared by different components of cloud infrastructure system 1102 and which facilitate the provisioning of services by cloud infrastructure system 1102. These internal shared services may include, without limitation, a security and identity service, an integration service, an enterprise repository service, an enterprise manager service, a virus scanning and whitelist service, a high availability, backup and recovery service, service for enabling cloud support, an email service, a notification service, a file transfer service, and the like.

Cloud infrastructure system 1102 may comprise multiple subsystems. These subsystems may be implemented in software, or hardware, or combinations thereof. As depicted in FIG. 11, the subsystems may include a user interface subsystem 1112 that enables users of cloud infrastructure system 1102 to interact with cloud infrastructure system 1102. User interface subsystem 1112 may include various interfaces such as a web UI 1114, an online store UI 1116 where cloud services provided by the cloud infrastructure system 1102 are advertised and are purchasable by a consumer, and other UIs 1118. For example, a tenant may, using a client device, request (service request 1134) one or more services provided by cloud infrastructure system 1102 using one or more interfaces 1114, 1116, and 1118. For example, a tenant may access the online store, browse cloud services offered by cloud infrastructure system 1102, and place a subscription order for one or more services offered by cloud infrastructure system 1102 that the tenant wishes to subscribe to. The service request may include information identifying the tenant and one or more services that the tenant desires to subscribe to. For example, a tenant may place a subscription order for a chatbot related service offered by cloud infrastructure system 1102. As part of the order, the client may provide information identifying the input (e.g., utterances).

In certain aspects, such as the embodiment depicted in FIG. 11, cloud infrastructure system 1102 may comprise an order management subsystem (OMS) 1120 that is configured to process the new order. As part of this processing, the OMS 1120 may be configured to: create an account for the tenant, if not done already; receive billing and/or accounting information from the tenant that is to be used for billing the tenant for providing the requested service to the tenant; verify the tenant information; upon verification, book the order for the tenant; and orchestrate various workflows to prepare the order for provisioning.

Once properly validated, OMS 1120 may then invoke the order provisioning subsystem (OPS) 1124 that is configured to provide resources for the order including processing, memory, and networking resources. The provisioning may include allocating resources for the order and configuring the resources to facilitate the service requested by the tenant order. The way resources are provisioned for an order and the type of the provisioned resources may depend upon the type of cloud service that has been ordered by the tenant. For example, according to one workflow, OPS 1124 may be configured to determine the cloud service being requested and identify several pods that may have been pre-configured for that cloud service. The number of pods that are allocated for an order may depend upon the size/amount/level/scope of the requested service. For example, the number of pods to be allocated may be determined based upon the number of users to be supported by the service, the duration of time for which the service is being requested, and the like. The allocated pods may then be customized for the requested tenant to provide the requested service.

Cloud infrastructure system 1102 may send a response or notification 1144 to the tenant to indicate when the requested service is now ready for use. In some instances, information (e.g., a link) may be sent to the tenant that enables the tenant to start using the benefits of the services requested.

Cloud infrastructure system 1102 may provide services to multiple tenants. For each tenant, cloud infrastructure system 1102 is responsible for managing information related to one or more subscription orders received from the tenant, maintaining tenant data related to the orders, and providing the requested services to the tenant or clients of the tenant. Cloud infrastructure system 1102 may also collect usage statistics regarding a tenant's use of subscribed services. For example, statistics may be collected for storage used, the amount of data transferred, the number of users, and the amount of system up time and system down time, and the like. This usage information may be used to bill the tenant. Billing may be done, for example, on a monthly cycle.

Cloud infrastructure system 1102 may provide services to multiple tenants in parallel. Cloud infrastructure system 1102 may store information for these tenants, including proprietary information. In certain aspects, cloud infrastructure system 1102 comprises an identity management subsystem (IMS) 1128 that is configured to manage tenant's information and provide the separation of the managed information such that information related to one tenant is not accessible by another tenant. IMS 1128 may be configured to provide various security-related services such as identity services, such as information access management, authentication and authorization services, services for managing tenant identities and roles and related capabilities, and the like.

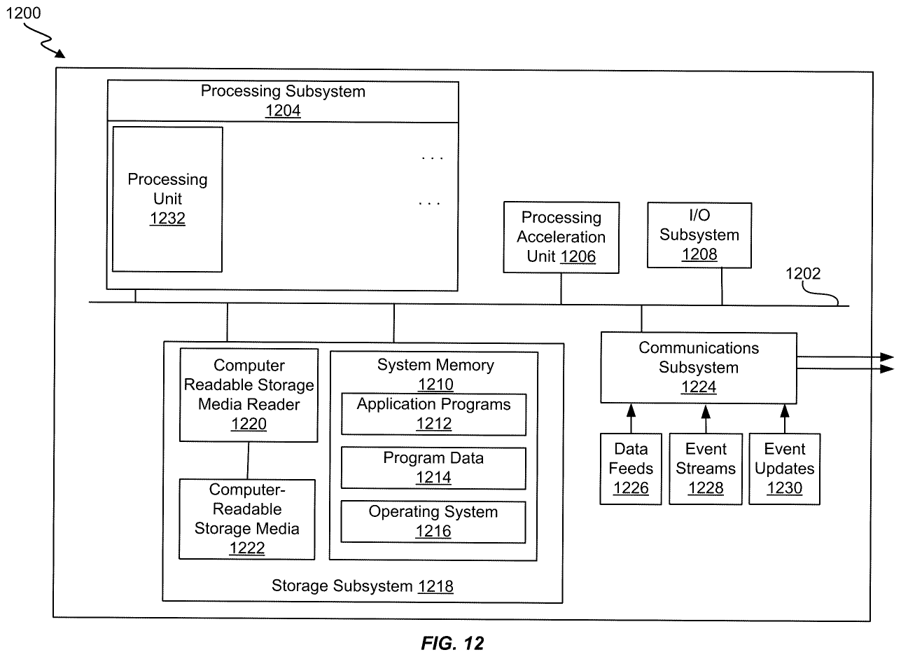

FIG. 12 illustrates a computer system 1200 that may be used to implement certain aspects. As shown in FIG. 12, computer system 1200 includes various subsystems including a processing subsystem 1204 that communicates with several other subsystems via a bus subsystem 1202. These other subsystems may include a processing acceleration unit 1206, an I/O subsystem 1208, a storage subsystem 1218, and a communications subsystem 1224. Storage subsystem 1218 may include a computer-readable storage media reader 1220, a computer-readable storage media 1222, and a system memory 1210.

Bus subsystem 1202 provides a mechanism for letting the various components and subsystems of computer system 1200 communicate with each other as intended. Although bus subsystem 1202 is shown schematically as a single bus, alternative aspects of the bus subsystem may utilize multiple buses. Bus subsystem 1202 may be any of several types of bus structures including a memory bus or memory controller, a peripheral bus, a local bus using any of a variety of bus architectures, and the like. For example, such architecture may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P13126.1 standard, and the like.

Processing subsystem 1204 controls the operation of computer system 1200 and may comprise one or more processors, application specific integrated circuits (ASICs), or field programmable gate arrays (FPGAs). The processors may be single core or multicore processors. The processing resources of computer system 1200 can be organized into one or more processing units 1232, 1234, etc. A processing unit may include one or more processors, one or more cores from the same or different processors, a combination of cores and processors, or other combinations of cores and processors. In some aspects, processing subsystem 1204 can include one or more special purpose co-processors such as graphics processors, digital signal processors (DSPs), or the like. In some aspects, some or all the processing units of processing subsystem 1204 can be implemented using customized circuits, such as application specific integrated circuits (ASICs), or field programmable gate arrays (FPGAs).

In some aspects, the processing units in processing subsystem 1204 can execute instructions stored in system memory 1210 or on computer-readable storage media 1222. In various aspects, the processing units can execute a variety of programs or code instructions and can maintain multiple concurrently executing programs or processes. At any given time, some or all the program code to be executed can be resident in system memory 1210 and/or on computer-readable storage media 1222 including potentially on one or more storage devices. Through suitable programming, processing subsystem 1204 can provide various functionalities described above. In instances where computer system 1200 executes one or more virtual machines, one or more processing units may be allocated to each virtual machine.

In certain aspects, a processing acceleration unit 1206 may optionally be provided for performing customized processing or for off-loading some of the processing performed by processing subsystem 1204 to accelerate the overall processing performed by computer system 1200.

I/O subsystem 1208 may include devices and mechanisms for inputting information into computer system 1200 and/or for outputting information from or via computer system 1200. In general, use of the term input device is intended to include all types of devices and mechanisms for inputting information into computer system 1200. User interface input devices may include, for example, a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may also include motion sensing and/or gesture recognition devices such as the Meta Quest® controller, Microsoft Kinect® motion sensor, the Microsoft Xbox® 360 game controller, or devices that provide an interface for receiving input using gestures and spoken commands. User interface input devices may also include eye gesture recognition devices such as a blink detector that detects eye activity (e.g., “blinking” while taking pictures and/or making a menu selection) from users and transforms the eye gestures as inputs to an input device. Additionally, user interface input devices may include voice recognition sensing devices that enable users to interact with voice recognition systems (e.g., Siri® navigator or Amazon Alexa®) through voice commands.

Other examples of user interface input devices include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, QR code readers, barcode readers, 3D printers, laser rangefinders, and eye gaze tracking devices. Additionally, user interface input devices may include, for example, medical imaging input devices such as computed tomography, magnetic resonance imaging, position emission tomography, and medical ultrasonography devices. User interface input devices may also include, for example, audio input devices such as MIDI keyboards, digital musical instruments, and the like.

In general, the use of the term output device is intended to include all types of devices and mechanisms for outputting information from computer system 1200 to a user or other computer. User interface output devices may include a display subsystem, indicator lights, or non-visual displays such as audio output devices, etc. The display subsystem may be any device for outputting a digital picture. Example display devices include flat panel display devices such as those using a light emitting diode (LED) display, a liquid crystal display (LCD) or plasma display, a projection device, a touch screen, a desktop or laptop computer monitor, and the like. As another example, wearable display devices such as Meta Quest® or Microsoft HoloLens® may be mounted to the user for displaying information. User interface output devices may include, without limitation, a variety of display devices that visually convey text, graphics, and audio/video information such as monitors, printers, speakers, headphones, automotive navigation systems, plotters, voice output devices, and modems.

Storage subsystem 1218 provides a repository or data store for storing information and data that is used by computer system 1200. Storage subsystem 1218 provides a tangible non-transitory computer-readable storage medium for storing the basic programming and data constructs that provide the functionality of some aspects. Storage subsystem 1218 may store software (e.g., programs, code modules, instructions) that when executed by processing subsystem 1204 provides the functionality described above. The software may be executed by one or more processing units of processing subsystem 1204. Storage subsystem 1218 may also provide a repository for storing data used in accordance with the teachings of this disclosure.

Storage subsystem 1218 may include one or more non-transitory memory devices, including volatile and non-volatile memory devices. As shown in FIG. 12, storage subsystem 1218 includes a system memory 1210 and a computer-readable storage media 1222. System memory 1210 may include several memories including a volatile main random-access memory (RAM) for storage of instructions and data during program execution and a non-volatile read only memory (ROM) or flash memory in which fixed instructions are stored. In some implementations, a basic input/output system (BIOS), containing the basic routines that help to transfer information between elements within computer system 1200, such as during start-up, may typically be stored in the ROM. The RAM typically contains data and/or programming modules that are presently being operated and executed by processing subsystem 1204. In some implementations, system memory 1210 may include multiple several types of memory, such as static random-access memory (SRAM), dynamic random-access memory (DRAM), and the like.

By way of example, and not limitation, as depicted in FIG. 12, system memory 1210 may load application programs 1212 that are being executed, which may include various applications such as Web browsers, mid-tier applications, relational database management systems (RDBMS), etc., program data 1214, and an operating system 1216. By way of example, operating system 1216 may include various versions of Microsoft Windows®, Apple Macintosh®, and/or Linux® operating systems, a variety of commercially-available UNIX® or UNIX-like operating systems (including without limitation the variety of GNU/Linux operating systems, the Oracle Linux®, Google Chrome® OS, and the like) and/or mobile operating systems such as iOS, Windows® Phone, Android® OS, and others.

Computer-readable storage media 1222 may store programming and data constructs that provide the functionality of some aspects. Computer-readable storage media 1222 may provide storage of computer-readable instructions, data structures, programming modules, and other data for computer system 1200. Software (programs, code modules, instructions) that, when executed by processing subsystem 1204 provides the functionality described above, may be stored in storage subsystem 1218. By way of example, computer-readable storage media 1222 may include non-volatile memory such as a hard disk drive, a magnetic disk drive, an optical disk drive such as a CD ROM, digital video disc (DVD), a Blu-Ray® disk, or other optical media. Computer-readable storage media 1222 may include Zip® drives, flash memory cards, universal serial bus (USB) flash drives, secure digital (SD) cards, DVD disks, digital video tape, and the like. Computer-readable storage media 1222 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, dynamic random access memory (DRAM)-based SSDs, magneto-resistive RAM (MRAM) SSDs, and hybrid SSDs that use a combination of DRAM and flash memory based SSDs.

In certain aspects, storage subsystem 1218 may also include a computer-readable storage media reader 1220 that can further be connected to computer-readable storage media 1222. The computer-readable storage media reader 1220 may receive and be configured to read data from a memory device such as a disk, a flash drive, etc.

In certain aspects, computer system 1200 may support virtualization technologies, including virtualization of processing and memory resources. For example, computer system 1200 may provide support for executing one or more virtual machines. In certain aspects, computer system 1200 may execute a program such as a hypervisor that facilitates the configuration and management of the virtual machines. Each virtual machine may be allocated memory, computer (e.g., processors, cores), I/O, and networking resources. Each virtual machine runs independently of the other virtual machines. A virtual machine typically runs its own operating system, which may be the same as or different from the operating systems executed by other virtual machines executed by computer system 1200. Accordingly, multiple operating systems may potentially be run concurrently by computer system 1200.

Communications subsystem 1224 provides an interface to other computer systems and networks. Communications subsystem 1224 serves as an interface for receiving data from and transmitting data to other systems from computer system 1200. For example, communications subsystem 1224 may enable computer system 1200 to establish a communication channel to one or more client devices via the Internet for receiving and sending information from and to the client devices. For example, the communications subsystem may be used to transmit a response to a user regarding the inquiry for a chatbot.

Communications subsystem 1224 may support both wired and/or wireless communication protocols. For example, in certain aspects, communications subsystem 1224 may include radio frequency (RF) transceiver components for accessing wireless voice and/or data networks (e.g., using cellular telephone technology, advanced data network technology, such as 3G, 4G or EDGE (enhanced data rates for global evolution), Wi-Fi (IEEE 1202.XX family standards, or other mobile communication technologies, or any combination thereof), global positioning system (GPS) receiver components, and/or other components. In some aspects, the communications subsystem 1224 can provide wired network connectivity (e.g., Ethernet) in addition to or instead of a wireless interface.

Communications subsystem 1224 can receive and transmit data in various forms. For example, in some aspects, in addition to other forms, communications subsystem 1224 may receive input communications in the form of data feeds 1226 that may be structured and/or unstructured, event streams 1228, event updates 1230, and the like. For example, communications subsystem 1224 may be configured to receive (or send) data feeds 1226 in real-time from users of social media networks and/or other communication services such as Twitter® feeds, Facebook® updates, web feeds such as Rich Site Summary (RSS) feeds, and/or real-time updates from one or more third party information sources.

In certain aspects, communications subsystem 1224 may be configured to receive data in the form of continuous data streams, which may include event streams 1228 of real-time events and/or event updates 1230, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1224 may also be configured to communicate data from computer system 1200 to other computer systems or networks. The data may be communicated in various forms such as structured and/or unstructured data feeds, event streams 1228, event updates 1230, and the like to one or more databases that may be in communication with one or more streaming data source computers coupled to computer system 1200.

Computer system 1200 can be one of various types, including a handheld portable device (e.g., an iPhone® cellular phone, an iPad® computing tablet, a personal digital assistant (PDA)), a wearable device (e.g., a Meta Quest® head mounted display), a personal computer, a workstation, a mainframe, a kiosk, a server rack, or any other data processing system. Due to the ever-changing nature of computers and networks, the description of computer system 1200 depicted in FIG. 12 is intended only as a specific example. Many other configurations having more or fewer components than the system depicted in FIG. 12 are possible. Based on the disclosure and teachings provided herein, a person of ordinary skill in art can appreciate other ways and/or methods to implement the various aspects.

Although specific aspects have been described, various modifications, alterations, alternative constructions, and equivalents are possible. Embodiments are not restricted to operation within certain specific data processing environments but are free to operate within a plurality of data processing environments. Additionally, although certain aspects have been described using a particular series of transactions and steps, it should be apparent to those skilled in the art that this is not intended to be limiting. Although some flowcharts describe operations as a sequential process, many of the operations can be performed in parallel or concurrently. In addition, the order of the operations may be rearranged. A process may have additional steps not included in the figure. Various features and aspects of the above-described aspects may be used individually or jointly.

Further, while certain aspects have been described using a particular combination of hardware and software, it should be recognized that other combinations of hardware and software are also possible. Certain aspects may be implemented only in hardware, or only in software, or using combinations thereof. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation, such as by executing computer instructions or code, or processors or cores programmed to execute code or instructions stored on a non-transitory memory medium, or any combination thereof. Processes can communicate using a variety of techniques including conventional techniques for inter-process communications, and different pairs of processes may use different techniques, or the same pair of processes may use different techniques at various times.

Specific details are given in this disclosure to provide a thorough understanding of the aspects. However, aspects may be practiced without these specific details. For example, well-known circuits, processes, algorithms, structures, and techniques have been shown without unnecessary detail to avoid obscuring the aspects. This description provides example aspects only, and is not intended to limit the scope, applicability, or configuration of other aspects. Rather, the preceding description of the aspects can provide those skilled in the art with an enabling description for implementing various aspects. Various changes may be made in the function and arrangement of elements.

The specifications and drawings are, accordingly, to be regarded in an illustrative rather than a restrictive sense. It can, however, be evident that additions, subtractions, deletions, and other modifications and changes may be made thereunto without departing from the broader spirit and scope as set forth in the claims. Thus, although specific aspects have been described, these are not intended to be limiting. Various modifications and equivalents are within the scope of the following claims.

1. A computer-implemented method comprising:
receiving, via a user interface, a request from a user including user-specific data in a natural language;
parsing the user-specific data from a current state of the user interface, wherein the current state indicates a chatbot-interaction between the user and an artificial intelligence (AI) agent at a given instant of time;
predicting a context of the request using one or more pre-historical logs;
utilizing the one or more pre-historical logs and a plurality of large language model (LLM) nodes to collect information of the user;
identifying from a data source, based on the context and information of the user, a plurality of resources that correspond to the request;
verifying availability of the plurality of resources for a time frame;
determining, based on the verification, a schedule of the request using a machine-learning engine;
retrieving a plurality of application programming interfaces (APIs) corresponding to the plurality of resources from a meta database to schedule the request;
automating scheduling of the request using the plurality of APIs;
calling an execution tool of a plurality of execution tools associated with the plurality of LLM nodes to modify the current state of the user interface; and
generating a new state at the user interface, wherein the new state includes an AI generated chat response associated with the request, a list of flags, and a plurality of mediated errors.

2. The computer-implemented method of claim 1, wherein the computer-implemented method is modeled as the AI agent along with an agent provider service to provide a plurality of services to the AI agent and maintain the context between graph executions.

3. The computer-implemented method of claim 1, wherein the AI agent involves a static directed cyclic graph that comprises the plurality of LLM nodes that are executed in parallel to manage request processing.

4. The computer-implemented method of claim 1, wherein the AI agent plans scheduling of the request and dynamically changes the schedule of the request based on a plurality of parameters.

5. The computer-implemented method of claim 1, wherein the plurality of LLM nodes comprises an intelligence node of a plurality of intelligence nodes that invokes the machine-learning engine one or more times, and an execution node of a plurality of execution nodes to call the execution tool of the plurality of execution tools that executes scheduling of the request in parallel.

6. The computer-implemented method of claim 1, wherein the data source includes the plurality of resources and provides data exchange across one or more organizations over a cloud network.

7. The computer-implemented method of claim 1, wherein a follow-up request is made at the user interface to confirm scheduling actions upon receiving a complex request.

8. A system comprising:
one or more processors;
one or more non-transitory computer-readable media storing instructions, which, when executed by the system, cause the system to perform a set of actions including:
parsing the user-specific data from a current state of the user interface, wherein the current state indicates a chatbot-interaction between the user and an artificial intelligence (AI) agent at a given instant of time;
predicting a context of the request using one or more pre-historical logs;
utilizing the one or more pre-historical logs and a plurality of large language model (LLM) nodes to collect information of the user;
identifying from a data source, based on the context and information of the user, a plurality of resources that correspond to the request;
verifying availability of the plurality of resources for a time frame;
determining, based on the verification, a schedule of the request using a machine-learning engine;
retrieving a plurality of application programming interfaces (APIs) corresponding to the plurality of resources from a meta database to schedule the request;
automating scheduling of the request using the plurality of APIs;
calling an execution tool of a plurality of execution tools associated with the plurality of LLM nodes to modify the current state of the user interface; and
generating a new state at the user interface, wherein the new state includes an AI generated chat response associated with the request, a list of flags, and a plurality of mediated errors.

9. The system of claim 8, wherein the set of actions further includes a plurality of services to the AI agent and maintaining the context between the graph executions.

10. The system of claim 8, wherein the AI agent involves a static directed cyclic graph that comprises the plurality of LLM nodes that are executed in parallel to manage request processing.

11. The system of claim 8, wherein the AI agent plans scheduling of the request and dynamically changes the schedule of the request based on a plurality of parameters.

12. The system of claim 8, wherein the plurality of LLM nodes comprises an intelligence node of a plurality of intelligence nodes that invokes the machine-learning engine one or more times, and an execution node of a plurality of execution nodes to call the execution tool of the plurality of execution tools that executes scheduling of the request in parallel.

13. The system of claim 8, wherein the data source includes the plurality of resources and provides data exchange across one or more organizations over a cloud network.

14. A computer-program product tangibly embodied in a non-transitory machine-readable storage medium, including instructions configured to cause one or more data processors to perform a set of actions including:
parsing the user-specific data from a current state of the user interface, wherein the current state indicates a chatbot-interaction between the user and an artificial intelligence (AI) agent at a given instant of time;
predicting a context of the request using one or more pre-historical logs;
utilizing the one or more pre-historical logs and a plurality of large language model (LLM) nodes to collect information of the user;
identifying from a data source, based on the context and information of the user, a plurality of resources that correspond to the request;
verifying availability of the plurality of resources for a time frame;
determining, based on the verification, a schedule of the request using a machine-learning engine;
retrieving a plurality of application programming interfaces (APIs) corresponding to the plurality of resources from a meta database to schedule the request;
automating scheduling of the request using the plurality of APIs;
calling an execution tool of a plurality of execution tools associated with the plurality of LLM nodes to modify the current state of the user interface; and
generating a new state at the user interface, wherein the new state includes an AI generated chat response associated with the request, a list of flags, and a plurality of mediated errors.

15. The computer-program product of claim 14, wherein the set of actions further includes a plurality of services to the AI agent and maintaining the context between the graph executions.

16. The computer-program product of claim 14, wherein the AI agent involves a static directed cyclic graph that comprises the plurality of LLM nodes that are executed in parallel to manage request processing.

17. The computer-program product of claim 14, wherein the AI agent plans scheduling of the request and dynamically changes the schedule of the request based on a plurality of parameters.

18. The computer-program product of claim 14, wherein the plurality of LLM nodes comprises an intelligence node of a plurality of intelligence nodes that invokes the machine-learning engine one or more times, and an execution node of a plurality of execution nodes to call the execution tool of the plurality of execution tools that executes scheduling of the request in parallel.

19. The computer-program product of claim 14, wherein the data source includes the plurality of resources and provides data exchange across one or more organizations over a cloud network.

20. The computer-program product of claim 14, wherein a follow-up request is made at the user interface to confirm scheduling actions upon receiving a complex request.
